(19) United States
(12) Patent Application Publication
(10) Pub. No.: US 2025/0375768 A1
(43) Pub. Date: Dec. 11, 2025

(54) CELL CULTURE DEVICES AND RELATED METHODS

(71) Applicant: STEMCELL TECHNOLOGIES CANADA INC., Vancouver (CA)

(72) Inventors: Douglas Kondro, Vancouver (CA); Michael Hyatt, Vancouver (CA)

(21) Appl. No.: 19/107,177

(22) PCT Filed: Sep. 8, 2023
(86) PCT No.: PCT/CA2023/051194

§ 371 Date: Feb. 27, 2025

Related U.S. Application Data
(60) Provisional application No. 63/405,040, filed on Sep. 9, 2022.

(51) Int. Cl. B01L 3/00; C12M 1/00; C12M 1/04; C12M 3/00

(52) U.S. Cl. CPC B01L 3/50853; B01L 3/502761; C12M 23/24; C12M 23/34; C12M 23/38; C12M 23/48; C12M 29/04; B01L 2200/0689; B01L 2300/0893; B01L 2400/0472

(57) ABSTRACT

This disclosure relates to laboratory devices and systems. methods of use, and methods of manufacture. Laboratory devices of this disclosure include a chamber or receptacle, and may also include one or more ports fluidically connected to the chamber. Thus. laboratory devices may be closed systems, and may therefore be particularly amenable to cell culture applications. In some embodiments. laboratory devices may include a gas permeable membrane. Liquid filling of and withdrawal from a chamber/receptacle of a laboratory device of this disclosure may be facilitated by a lid or adapter feature that imparts a tilt or angle to the chamber.

### CROSS REFERENCE TO RELATED APPLICATION

This application claims the benefit of U.S. Provisional Patent Application No. 63/405,040, filed Sep. 9, 2022, the entire content of which is hereby incorporated by reference in its entirety.

### TECHNICAL FIELD

This disclosure relates to laboratory devices, such as for culturing, incubating or aggregating cells. More specifically this disclosure relates to laboratory devices for culturing, incubating or aggregating cells at scale.

### BACKGROUND

Two dimensional (2D) culture of adherent cells in a monolayer sheet, such as using T-flasks, has been the gold standard. Standard equipment has been developed to allow users to efficiently grow cells in a dish or well plate format at a relatively low cost. In theory, cells grown in a 2D monolayer receive uniform amounts of nutrients and growth factors, and they can be easily lifted from their growth surface.

Several limitations exist for 2D cell cultures. For example, the formation of a monolayer leads to reduced cell-to-cell interactions. In addition, plastic surfaces used to support monolayer culture are much stiffer than an in vivo environment. While the use of hydrogels may mitigate some of the effects of culturing cells on plastic, they do not completely obviate this issue.

In contrast, three-dimensional (3D) culture may be a format that better recapitulates in vivo conditions during in vitro culture for many cell types. In comparison to 2D culture, cells grown in 3D experience enhanced cell-to-cell and cell-to-extracellular matrix interactions. Improved gene expression, cell junction formation, differentiation and drug response may be other advantages for certain cell types in 3D cultures.

Nevertheless, several disadvantages exist among current 3D culture systems, such as poor reproducibility, higher cost, and increased experimental complexity.

Different formats are used for 3D culture, including scaffold-based assemblies and cell-based assemblies. In scaffold-based assemblies, cells associate with a non-cellular substrate, such as embedded in a hyrdrogel or a porous biomaterial. In cell-based assemblies, cells may spontaneously assemble due to cell-to-cell affinity to form cellular aggregates. 3D cultured aggregates have been used in a wide range of applications, including expansion, modeling, drug screening, and tissue delivery.

Considering the advantages of 3D culture systems, there exists a need to develop cost-effective systems, devices and methods that reproducibly realize these advantages while overcoming the various disadvantages of current systems and devices.

### SUMMARY

In one aspect of this disclosure is provided a laboratory device. A laboratory device of this aspect may comprise a housing having one or more sidewalls extending substantially orthogonally from a planar member, and a gas permeable membrane in a sealed engagement with the housing, the gas permeable membrane and the housing forming a receptacle having a chamber defined by a top wall and a bottom wall that are connected and circumscribed by the one or more sidewalls.

A laboratory device of this disclosure may further comprise a first port and an opposed second port each in fluid communication with the chamber. In one embodiment, the first port and the second port are diagonally or diametrically opposed. In one embodiment, the first port and the second port extend through the top wall.

In one embodiment, the laboratory device is closed and/or sealed.

In one embodiment, a diameter of the second port is the same or larger than a diameter of the first port. In one embodiment, a diameter of the first port is between about 3 mm and 5 mm. In one embodiment, a diameter of the second port is between about 3 mm and 12 mm. In one embodiment, a diameter of the first port and a diameter of the second port are not the same.

A laboratory device of this disclosure may further comprise a plurality of micropatterned features in the bottom wall of the chamber. In one embodiment, the micropatterned features are cylindrical, inverted cones, inverted frustums of cones, inverted pyramids, or inverted frustums of pyramids. In one embodiment, a depth of each micropatterned feature is between about 100 μm to 4 mm. In one embodiment, a width or diameter of each micropatterned feature taken in the plane across an opening thereof is between about 100 μm to 5 mm. In one embodiment, an aspect ratio of each micropatterned feature is less than 1.

In one embodiment, the gas permeable membrane forms the bottom wall and the plurality of micropatterned features are formed in or on the gas permeable membrane.

In one embodiment, the gas permeable membrane forms the top wall and the planar member forms the bottom wall, and the plurality of micropatterned features are formed in or on the planar member.

A laboratory device of this disclosure may further comprise a frame external the chamber and overlapping at least a perimeter of the gas permeable membrane. In one embodiment, the frame comprises at least one brace against the gas permeable membrane to limit gas permeable membrane stretch and chamber volume increase when the chamber is filled with a fluid.

In one embodiment, the first port and the second port are formed in and/or traverse opposed corners or edges of the frame.

In one embodiment, the first port and the second port are respectively bounded by cooperating frame wall portions and a connecting wall portions, to form first and second port reservoirs. In one embodiment, a height of the connecting wall portions is lower than a height of the frame wall portions.

A laboratory device of this disclosure may further comprise a lid having a continuous skirt extending orthogonally downward from an upper plane thereof. In one embodiment, a height of the skirt at a first edge or corner of the lid is minimal and a height of the skirt at an opposed second edge or corner of the lid is maximal.

In one embodiment, the first edge or corner of the lid, the second edge or corner of the lid, the first port, and the second port lie along a common axis when viewed from above and when the lid is in a position covering the housing.

In one embodiment, the bottom wall of the receptacle is tilted when the housing is positioned on the lid and when the skirt rests on a level surface. In one embodiment, the bottom wall is tilted about a tilt axis that is orthogonal to the common axis. In one embodiment, the bottom wall is tilted between 0 and 45 degrees, and preferably less than 10 degrees, and more preferably 5 degrees or less.

In one embodiment, at least the frame and the housing are made from a polymer independently selected from polystyrene (PS), polymethylpentene (PMP), polycarbonate (PC), polymethyl methacrylate (PMMA), silicon, silicone-based, or a styrene block copolymer. In one embodiment, the gas permeable membrane is made from polystyrene (PS), polymethylpentene (PMP), polycarbonate (PC), polymethyl methacrylate (PMMA), silicon, silicone-based, or a styrene block copolymer.

In another aspect of this disclosure is provided a laboratory device. A laboratory device of this aspect may comprise a receptacle having one or more sidewalls extending substantially orthogonally upward from a bottom wall; one or more limits surrounding the one or more sidewalls, the one or more limits extending a non-constant and shorter distance from the bottom wall relative to the one or more sidewalls; and a lid having a continuous skirt extending orthogonally downward from an upper plane thereof, a height of the skirt at a first edge or corner of the lid is minimal and a height of the skirt at an opposed second edge or corner of the lid is maximal.

In one embodiment, the bottom wall lies in a substantially level plane when the receptacle rests on a level surface, and the upper plane of the lid lies in a plane that is parallel to the bottom wall when the skirt rests against the one or more limits, and the bottom wall is tilted relative to the level surface when an underside of the receptacle is positioned on the upper plane of the lid as the skirt rests against the level surface. In one embodiment, the bottom wall is tilted about a tilt axis that is orthogonal to an axis through the first edge or corner and the opposed second edge or corner of the lid (when viewed from above). In one embodiment, the bottom wall is tilted between 0 and 45 degrees, and preferably less than 10 degrees, and more preferably 5 degrees or less.

A laboratory device of this disclosure may further comprise a gas permeable membrane sealingly secured to the one or more sidewalls.

In one embodiment, the gas permeable membrane forms the bottom wall.

In one embodiment, the gas permeable membrane is spaced apart from and lies in a plane parallel to a plane of the bottom wall (e.g. the membrane forms the top wall).

A laboratory device of this disclosure may further comprise a plurality of micropatterned features in the bottom wall of the receptacle. In one embodiment, the micropatterned features are cylindrical, inverted cones, inverted frustums of cones, inverted pyramids, or inverted frustums of pyramids. In one embodiment, a depth of each micropatterned feature is between about 100 μm to 4 mm. In one embodiment, a width or diameter of each micropatterned feature taken in the plane across an opening thereof is between about 100 μm to 5 mm. In one embodiment, an aspect ratio of each micropatterned feature is less than 1.

A laboratory device of this disclosure may further comprise a first port and an opposed second port each in fluid communication with a chamber formed between the bottom wall and gas permeable membrane, and circumscribed by the one or more sidewalls. In one embodiment, a diameter of the second port is the same or larger than a diameter of the first port.

A laboratory device of this disclosure may further comprise a frame external the chamber and overlapping at least a perimeter of the gas permeable membrane. In one embodiment, the frame comprises at least one brace against the gas permeable membrane to limit gas permeable membrane stretch and chamber volume increase when the chamber is filled with a fluid.

In one embodiment, the first port and the second port traverse and/or are configured in opposed corners or edges of the frame. In one embodiment, the first port and the second port are respectively bounded by cooperating frame wall portions and connecting wall portions, forming first and second port reservoirs. In one embodiment, a height of the connecting wall portions is lower than a height of the frame wall portions.

In another aspect of this disclosure are provided methods of using devices of this disclosure in laboratory assays, experiments, or incubations. For example, the assays, experiments, or incubations may involve cells, or other types of analytes, such as biomolecules. In embodiments involving cells, methods may relate to culturing or incubating cells, such as to form unadhered aggregates of cells. Regardless, of the processes in which devices of this disclosure are used, addition and/or removal of liquids from a receptacle/chamber thereof may be facilitated by tilting the device, such as in cooperation with a provided lid. In certain embodiments, methods of this disclosure involved closed and/or sealed devices, in particular where the methods involve cells. In such embodiments, liquid (e.g. cell suspensions and/or culture media) may be introduced into a closed/sealed chamber via ports.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows various views of an exemplary device of this disclosure. Depicted are a perspective side view (A), a cross sectional view (B), and a top view (C).

FIG. 2 shows a perspective side view (A) and a cross-sectional view taken through the plane “A” (B) of a base/housing of one embodiment of a device of this disclosure.

FIG. 3 shows an exploded view (A) and a zoomed in exploded view (B) of different embodiments of a device of this disclosure, also highlighting potential methods of manufacturing.

FIG. 4 shows a perspective view (A) and a cross-sectional view (B) of a different embodiment of a device of this disclosure.

FIG. 5 shows cross-sectional views of various embodiments of micropatterned features.

FIG. 6 shows various embodiments of frames and braces comprised in devices of this disclosure (A) and respective impacts on the device chamber volume (B).

FIG. 7 shows different embodiments of ports in panels (A) and (B).

FIG. 8 shows images of fluid withdrawal operations of devices of this disclosure tilted at either 0° (A), 1° (B), 2° (C), or 3° (D).

FIG. 9 shows the relationship of a base/housing and lid of an exemplary device. A partial exploded view is shown in (A) with base/housing floating above the lid. Perspective (B), front (C) and side (D) views of base/housing tiltedly resting upon lid.

FIG. 10 shows the relationship of a base/housing and lid of an exemplary device. A partial exploded view is shown in (A) with lid floating above the base/housing. Perspective (B) and front (C) views of lid resting upon base/housing.

FIG. 11 shows a method of manufacturing a gas permeable membrane of this disclosure having a plurality of micropatterned features thermoformed therein.

### DETAILED DESCRIPTION

This disclosure relates to laboratory devices (e.g. cell culture devices), systems and methods related to their use or manufacture. Devices of this disclosure may be used to culture, incubate and/or aggregate cells. In some embodiments, laboratory devices comprise a micropatterned surface (e.g. a surface having a plurality of microwells). In one embodiment, laboratory devices comprise a closed or sealed chamber. In one embodiment, scale-out beyond the limits of a single laboratory device (e.g. cell culture device) may be achieved using a plurality of individual devices.

Where used in this disclosure, the term “laboratory device” refers to a device used in laboratories in which experiments or assays may be carried out, such as experiments or assays on liquids which may comprise analytes, biomolecules, or cells. In one embodiment, a laboratory device is a cell culture device. Thus, where used in this disclosure, the term “cell culture device” refers to a device into which cells may be seeded and incubated. Cells seeded into a cell culture device of this disclosure are not particularly limited, and may either be adherent cells or non-adherent cells. Cells placed into a chamber of the disclosed devices may be primary cells, cell lines, cancer cells, pluripotent stem cells, or cells differentiated from pluripotent stem cells, etc. In one embodiment, a chamber of a laboratory device, and in particular at least a surface thereof that is normal to the force of gravity, is not itself amenable to the 2D culture of a monolayer of adherent cells. In such an embodiment, ordinarily adherent cells seeded into the chamber may rather form suspended cell aggregates, embryoid bodies, or organoids. In one embodiment, at least one surface of an internal chamber or receptacle (normal to the force of gravity) is modified to include a plurality of micropatterned features (e.g. microwells), as further described below.

Where used in this disclosure, the terms “cell aggregate” or “aggregate” refers to a grouping of cells that have coalesced to form an interconnected mass. Cells may spontaneously form into an aggregate, or they may be urged to form an aggregate. A plurality of cells may be urged to coalesce into an aggregate when they are forced into direct contact. The formation of an aggregate can be influenced by positioning a plurality of cells against a surface topology. In embodiments, where the cells are adherent cells it will be important that their self-aggregation tendencies overcome their tendencies to adhere to a non-cellular surface, such as a cell culture surface.

Unless otherwise defined, scientific and technical terms used in connection with the devices, systems and methods described herein shall have the meanings that are commonly understood by those of ordinary skill in the art. The terminology used herein is for the purpose of describing particular embodiments only and is not intended to limit the scope of the present invention, which is defined solely by the claims.

### Devices and Systems

In one aspect of this disclosure are provided laboratory devices, such as cell culture devices. In one embodiment, devices of this disclosure are closed systems or sealed systems. In other words, an internal chamber of a device is not directly exposed to the external environment, but is rather sealed from the external environment. However, a closed cell culture device may include gas exchange means to introduce oxygen into an internal chamber thereof. Also, given the need for nutrients and/or growth factors of cells in culture, a closed cell culture device will include means for introducing nutrients and/or growth factors, preferably contained in a cell culture medium, into an internal chamber thereof.

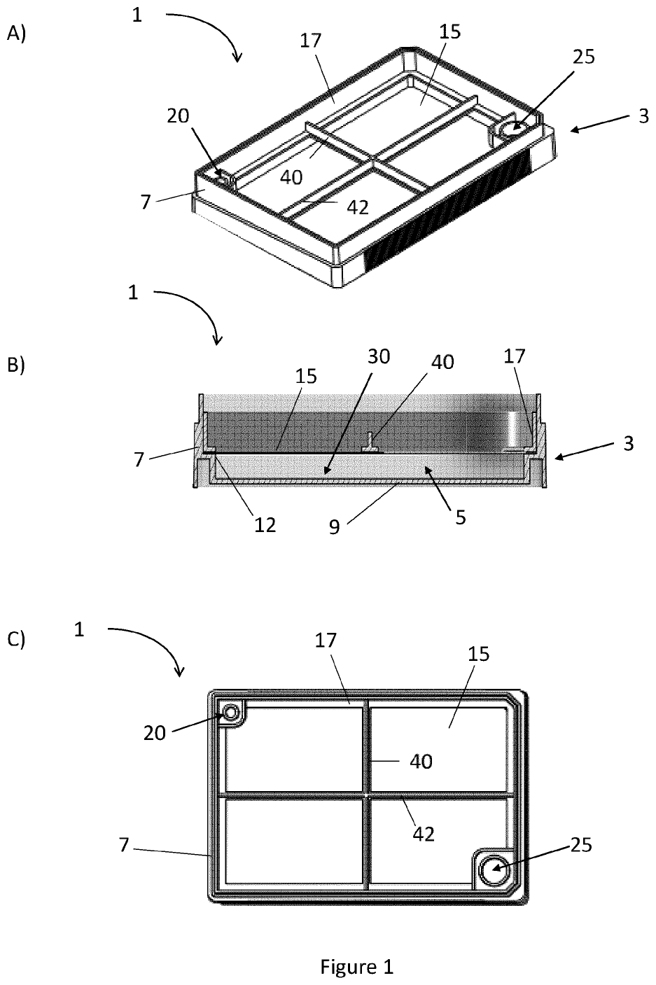

With reference to FIG. 1, a device 1 of this disclosure may comprise a housing 3 that defines or cooperates to define a receptacle and/or a chamber 5. Housing 3 may comprise one or more sidewalls 7 extending substantially orthogonally from a substantially planar member 9. In one embodiment, one or more sidewalls 7 and planar member 9 are integral. In one embodiment, one or more sidewalls 7 and planar member 9 are of at least two-piece construction.

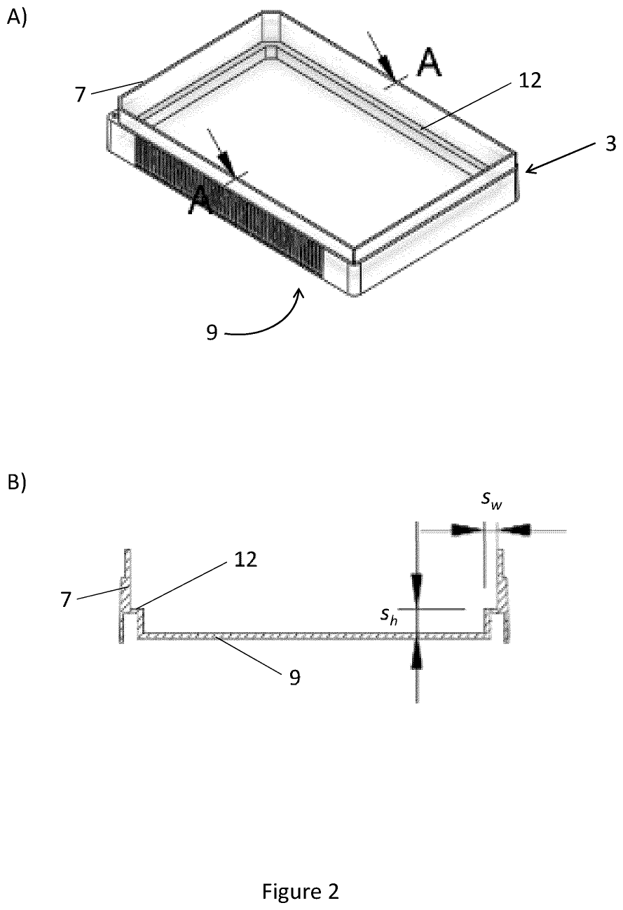

With reference to FIG. 1B and 2, housing 3 may comprise a first shoulder 12. First shoulder 12 extends orthogonally or substantially orthogonally away from one or more sidewall 7. More particularly, shoulder 12 may extend from a point that is intermediate the base and apex of one or more sidewalls 7 toward an interior of chamber/receptacle 5. In one embodiment, shoulder 12 is formed on or in an inner surface of one or more sidewalls 7 (e.g. a surface of one or more sidewalls on the chamber/receptacle side). In one embodiment, shoulder 12 forms a perimeter within chamber/receptacle 5.

Shoulder 12 can be any width s_{w}. For example, shoulder 12 provides sufficient surface area for an adhesive to be applied thereto, but is not so wide as to drastically reduce a volume of chamber/receptacle 5. In one embodiment, a width of shoulder 12 is about 1 mm. In one embodiment, a width of shoulder 12 is about 2 mm. In one embodiment, a width of shoulder 12 is about 3 mm. In one embodiment, a width of shoulder 12 is about 4 mm. In one embodiment, a width of shoulder 12 is about 5 mm. In a preferred embodiment, a width of shoulder 12 is between about 1 mm and 5 mm.

Shoulder 12 can be any height sₕ; but should be sufficient to hold a desired volume within receptacle/chamber 5. In one embodiment, a height of shoulder 12 is about 2 mm. A height of shoulder 12 (taken from bottom wall) may be about 4 mm, about 6 mm, about 8 mm, about 10 mm, about 12 mm, about 14 mm, about 16 mm, about 18 mm, or about 20 mm. In a preferred embodiment, a height of shoulder 12 (taken from bottom wall) is between about 5 mm and 20 mm.

Housing 3 may be made of any material, but preferably comprises a polymer. In one embodiment, housing 3 is made of a material amenable to molding technology, such as injection molding. Non-limiting examples of materials that housing 3 may be made of include: polystyrene (PS), polymethylpentene (PMP), polycarbonate (PC), polymethyl methacrylate (PMMA), silicon, silicone-based material, or a copolymer, such as a styrene block copolymer.

Device 1 may further comprise a gas permeable membrane 15. Gas permeable membrane 15 cooperates with housing 3 to form receptacle/chamber 5, which chamber 5 may be defined by a top wall and a bottom wall, which are connected and circumscribed by at least a portion of the one or more sidewalls 7. Gas permeable membrane 15 may be constructed of any material provided that oxygen and other gases readily diffuse therethrough (into receptacle/chamber 5) and that it is non-toxic or does not damage biomolecules or cells, or off-gas toxins or contaminants into receptacle/chamber 5.

Gas permeable membrane 15 may be made of any material, but preferably comprises a polymer. In one embodiment, gas permeable membrane 15 is made of a material amenable to extrusion or molding. Non-limiting examples of materials that gas permeable membrane 15 may be made of include: PS, PMP, PC, SBS/SEBS, silicon, silicone-based material, or a copolymer, such as a styrene block copolymer.

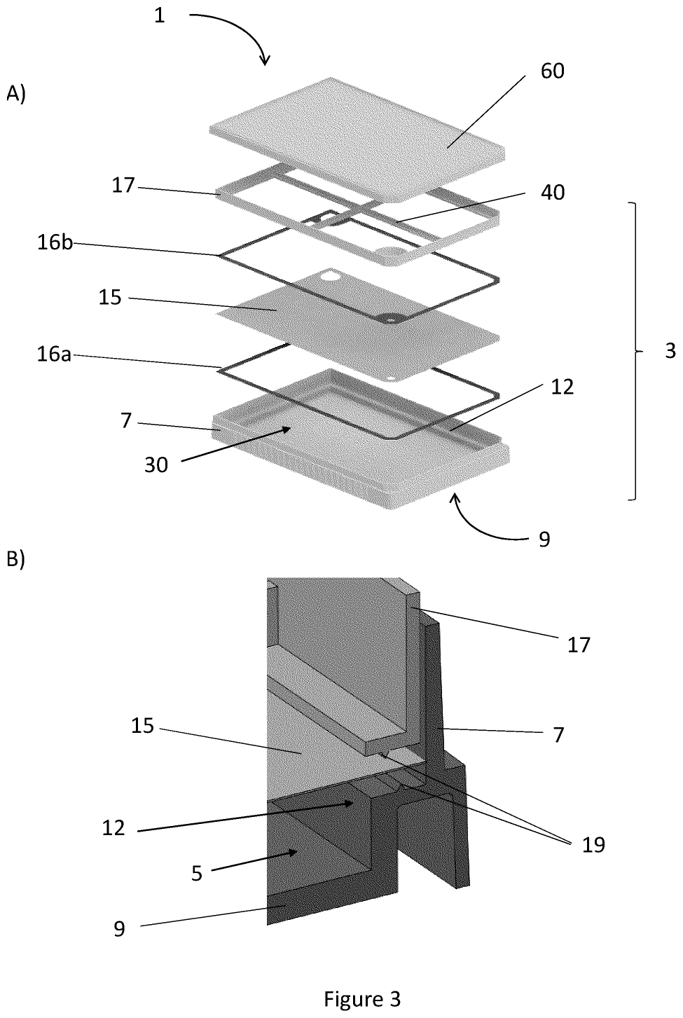

In one embodiment, gas permeable membrane 15 forms a top wall of chamber 5 (as depicted in FIGS. 1 and 3), and in such case bottom wall of chamber 5 may be planar member 9. Thus, gas permeable membrane 15 may be bonded or otherwise attached to one or more sidewalls 7, or more specifically to shoulder 12. Gas permeable membrane may be otherwise attached to housing 3, such as by any means known to the skilled artisan. In one embodiment, gas permeable membrane 15 is attached to housing 3 (e.g. one or more sidewalls 7, or shoulder 12) in such a way to ensure a sealed engagement (e.g. leak proof) under normal use conditions (e.g. incubation at 37°-75°). In addition, selection of the attachment agent may be important in terms of biocompatibility and/or the ability to attach/bond disparate materials.

In an exemplary embodiment depicted in FIG. 3A, an adhesive 16a is applied to shoulder 12 to secure gas permeable membrane 15 to housing 3. The adhesive may be any type of adhesive, provided that it is capable of adhering the materials that shoulder 12 and gas permeable membrane 15 are made of. In one embodiment, the adhesive is a double-sided tape. In one embodiment, the adhesive is a glue.

Device 1 may further comprise a frame 17. Frame 17 may provide one or more structural attributes and/or one or more functional attributes. Potential roles of frame 17 may include facilitating securement of gas permeable membrane 15 to housing 3 (such as to shoulder 12); stabilizing gas permeable membrane 15 from stretching as chamber 5 is filled with liquid, supporting/incorporating bores or ports through which liquid may be introduced or withdrawn from chamber 5. Thus, in one embodiment, frame 17 may cooperate with housing 3 (such as shoulder 12) and adhesives 16a and 16b to attach or secure gas permeable membrane 15.

In an exemplary embodiment depicted in FIG. 3B, gas permeable membrane 15 is secured to housing 3 by welding, such as by ultrasonic welding. Securing gas permeable membrane 15 to housing 3 by ultrasonic welding may be facilitated by frame 17 external chamber 5 (described further below) that is placed about or overlaps/overlies at least a perimeter of gas permeable membrane 15, such that membrane 15 is sandwiched between shoulder 12 and frame 17. In such embodiments, frame 17 and shoulder 12 may comprise cooperating ribs 19 that come into contact to further facilitate ultrasonic welding. In other embodiments, frame 17 and shoulder 12 may respectively comprise mateable ribs and grooves that cooperate to secure gas permeable membrane 15 to housing 3.

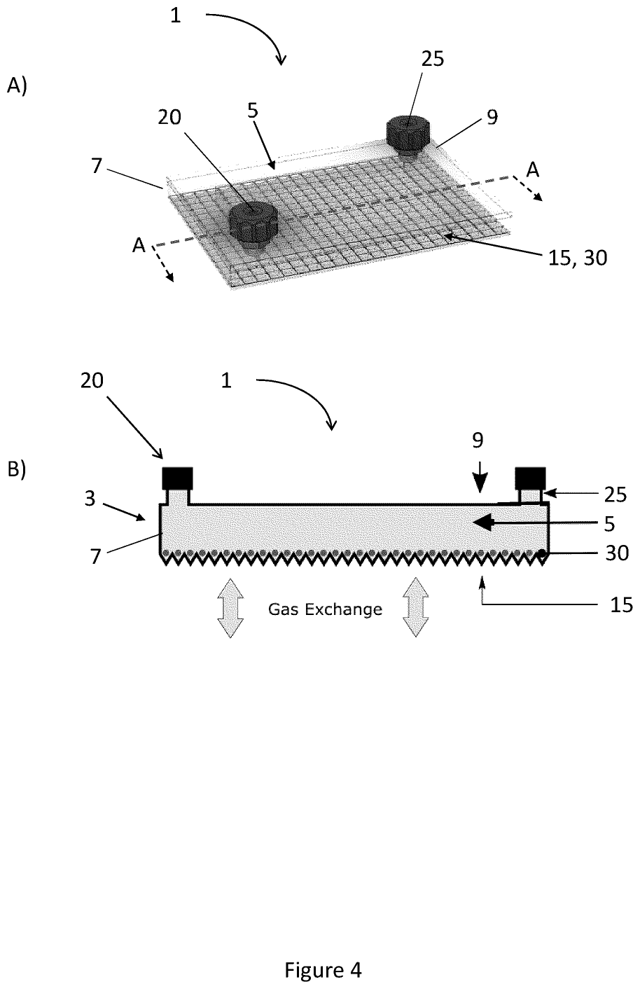

In one embodiment, gas permeable membrane 15 forms a bottom wall of chamber 5 (as depicted in FIG. 4), and in such case top wall may be planar member 9. Thus, gas permeable membrane 15 may be bonded or otherwise attached to one or more sidewalls 7. In one embodiment, gas permeable membrane 15 may be directly bonded or otherwise attached to a rim of one or more sidewalls 7. In one embodiment, a shoulder and/or a frame feature essentially as described above (except inverted) may mediate attachment of gas permeable membrane 15 to housing 3. In one embodiment, gas permeable membrane 15 is attached to housing 3 in such a way to ensure a sealed engagement (e.g. leak proof) under normal use conditions, and the means of attachment may be as described above or any other way known to skilled artisans. In addition, selection of the attachment agent may be important in terms of biocompatibility and/or the ability to attach/bond disparate materials.

Gas permeable membrane 15 is not particularly limited in terms of its dimensions, and more particularly its thickness, provided that gas can diffuse across the membrane to the same or better extent compared to materials from which microplates or cell culture flasks are made. In one embodiment, gas permeable membrane 7 is between about 0.05 mm and 1 mm thick. In one embodiment, gas permeable membrane 7 is between about 0.1 mm and 0.8 mm thick. In one embodiment, gas permeable membrane 7 is between about 0.15 mm and 0.7 mm thick. In one embodiment, gas permeable membrane 7 is between about 0.2 mm and 0.65 mm thick. In one embodiment, gas permeable membrane 7 is between about 0.25 mm and 0.6 mm thick. In one embodiment, gas permeable membrane 7 is about 0.2 mm thick, about 0.3 mm thick, about 0.4 mm thick, about 0.5 mm thick, about 0.6 mm thick, about 0.7 mm thick, about 0.8 mm think, or thicker.

In one embodiment, elements that define or cooperate to define chamber 5 may provide for different permeability of gases. In one embodiment, only gas permeable membrane 15 is permeable to gases or to sufficient quantities of gases over the time scale of (cell) cultures, incubations, or aggregations. More particularly, housing 3 may not be gas permeable or may only be permeable to insufficient quantities/volumes of gases over the time scale of (cell) cultures, incubations, or aggregations.

Device 1 may further comprise at least one port, and preferably more than one port. In one embodiment, device 1 comprises a first port 20 and a second port 25 (FIGS. 1 and 4). First port 20 and second port 25 are each in fluid communication with chamber 5, but may nevertheless be plugged or pluggable to prevent the escape of contents from chamber 5 and/or to protect the contents of chamber 5 from the environment external of device 1.

Positioning of first port 20 and/or second port 25 may depend on how chamber 5 is configured with respect to the localization of gas permeable membrane 15. For example, first port 20 and/or second port 25 may pass through a top wall of chamber 5 when gas permeable membrane 15 forms the bottom wall. By way of additional example, first port 20 and/or second port 25 may pass through gas permeable membrane 15, or respective apertures therein, when the membrane forms a top wall of chamber 5. In one embodiment, first port 20 and/or second port 25 may pass through one or more sidewalls 7.

In one embodiment, first port 20 and/or second port 25 lie over, or pass or extend through top wall (e.g. membrane 15 or planar member 9, depending on the configuration) of device 1. Thus, first port 20 and/or second port 25 cooperate with respective bores through top wall. In one embodiment, one or both ports may extend toward about 0.5 to 2 mm of bottom wall. In one embodiment, first port 20 and/or second port 25 may lie over, or extend or pass through one or more sidewall 7 of housing 3.

A diameter of first port 20 (and in some embodiments a diameter of a bore cooperating with ports) will not impede the passage of air therethrough, otherwise the diameter is not particularly limited. In one embodiment, a diameter of first port 20 (and in some embodiments a diameter of a bore cooperating with ports) is not less than 3 mm. In one embodiment, a diameter of first port 20 (and in some embodiments a diameter of a bore cooperating with ports) is between about 3 mm and 5 mm. In one embodiment, a diameter of first port 20 (and in some embodiments a diameter of a bore cooperating with ports) is about 4 mm.

A diameter of second port 25 (and in some embodiments a diameter of a bore cooperating with ports) will not impede the passage of a liquid, such as a cell culture medium. In one embodiment, a diameter of second port 25 (and in some embodiments a diameter of a bore cooperating with ports) is the same or larger than a diameter of first port 20. In one embodiment, a diameter of second port 25 (and in some embodiments a diameter of a bore cooperating with ports) is between about 3 mm and 12 mm. In one embodiment, a diameter of second port 25 (and in some embodiments a diameter of a bore cooperating with ports) is about 10 mm or about 12 mm. A relatively larger diameter (e.g. 10 mm or 12 mm) may be preferred when a serological pipette, or something similarly dimensioned, is used to introduce and withdraw fluid into or out of chamber 5. A relatively smaller diameter (e.g. between 3 mm-6 mm) may be preferred when a pipette smaller than a serological pipette is used or when tubing is connected to a pump is used to introduce and withdraw liquid into or out of chamber 5.

Ports included in device 1 may be made of any material. Commonly, ports are made of a type of polymer, which may lend to being thermoformable. In one embodiment, ports may be or comprise Luer fittings.

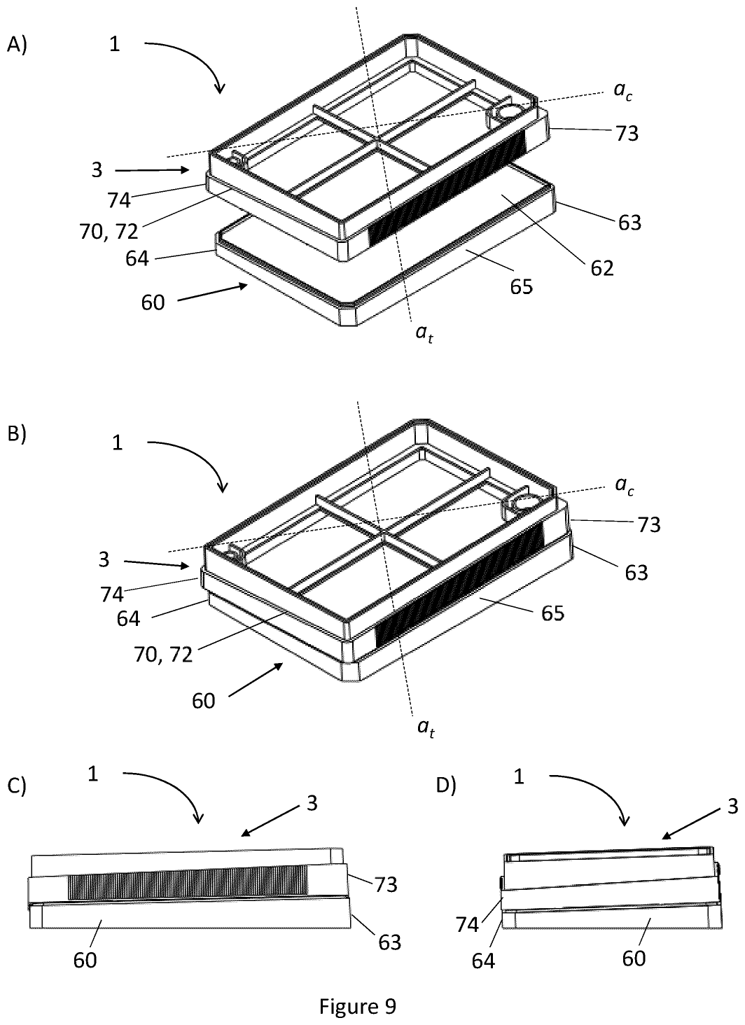

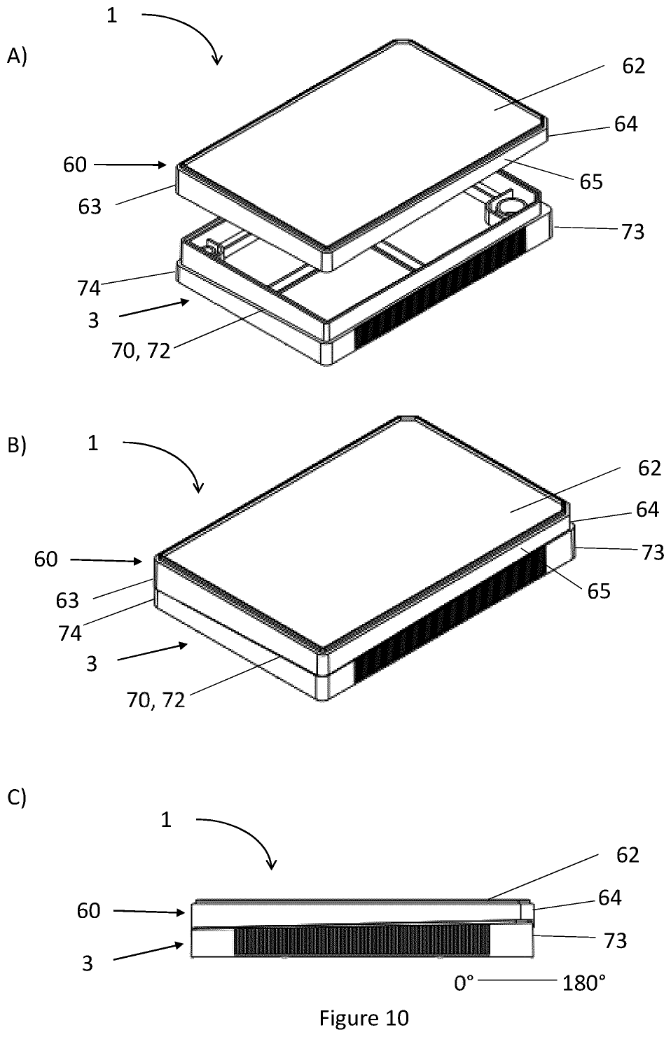

In preferred embodiments, device 1 comprises two ports (e.g. first port 20 and second port 25). In such embodiment, the ports may be positioned in or near opposed corners or edges of device 1 (e.g. elements 73 and 74 as shown in FIGS. 9 and 10). More specifically, the ports may be positioned in or near diagonally or diametrically opposed corners or edges of device 1, such as of top wall.

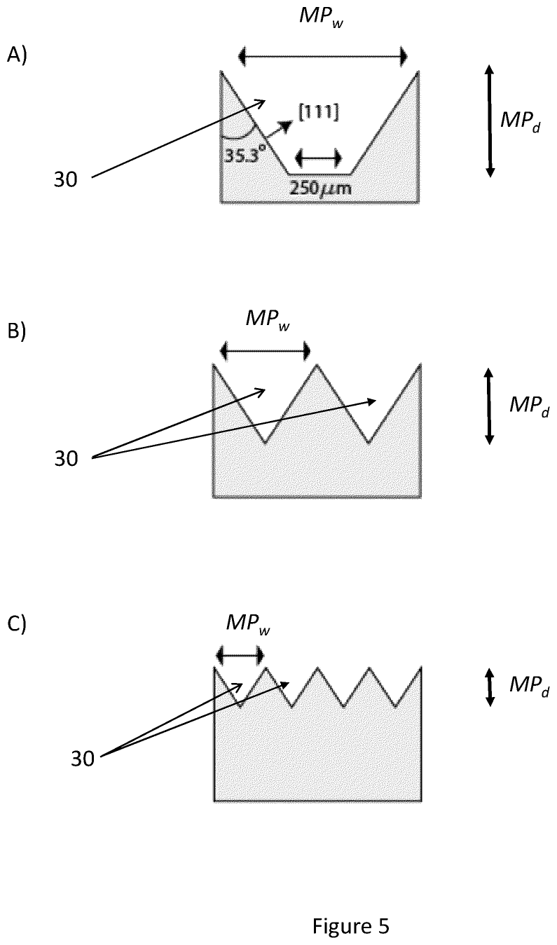

In one embodiment, device 1 further comprises a plurality of micropatterned features 30 (see FIGS. 4 and 5). Plurality of micropatterned features 30 may be dimensioned to receive a plurality of cells. Thus, in some embodiments, plurality of micropatterned features 30 are formed in or disposed on an internal surface of chamber 5 that is normal to the force of gravity, such as bottom wall (e.g. planar member or gas permeable membrane depending on the configuration). In one embodiment, cells received into a respective micropatterned feature will coalesce into an (unadhered) aggregate of cells. In one embodiment, bottom wall of chamber 5 in or on which plurality of micropatterned features are formed does not itself support anchorage dependent growth of the cells.

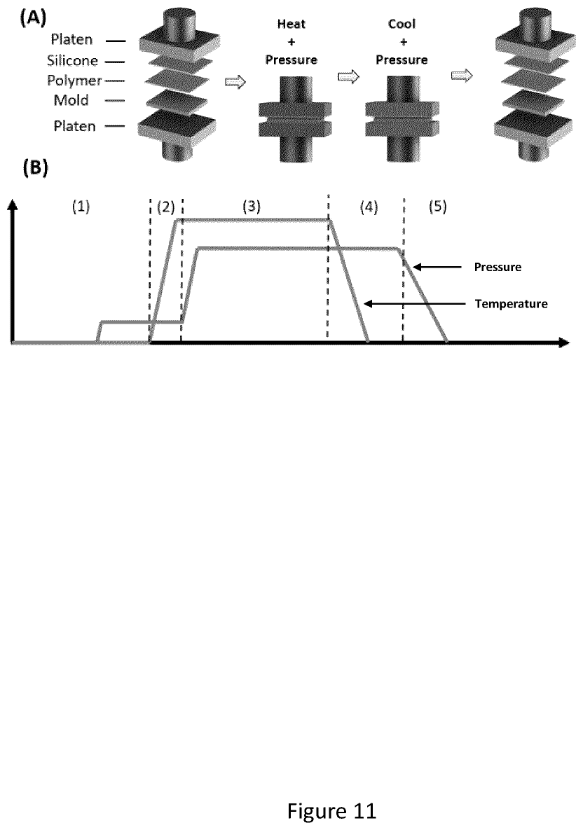

In embodiments where gas permeable membrane 15 forms bottom wall of chamber 5, plurality of micropatterned features 30 may be formed in or on gas permeable membrane 15. Micropatterned gas permeable membranes may be manufactured using thermoforming methods, such as embossing (see FIG. 11, for example). In such embodiments, plurality of micropatterned features 30 may descend from an upper plane of gas permeable membrane 15.

In embodiments where gas permeable membrane 15 forms a top wall of chamber 5, plurality of micropatterned features 30 may be formed in or on bottom wall (e.g. planar member 9). Micropatterned bottom wall may be manufactured using thermoforming methods, such as embossing or liquid molding, or by stamping or etching. In such embodiments, plurality of micropatterned features 30 may descend from an upper plane of bottom wall (e.g. a base of housing 3).

Each micropatterned feature may be the same shape. In one embodiment, differently shaped features may be comprised within plurality of micropatterned features 30. Regardless, plurality of micropatterned features 30 may be cylindrical, inverted cones, inverted frustums of cones, inverted pyramids, or inverted frustums of pyramids. In a preferred embodiment, plurality of micropatterned features 30 are inverted pyramids or frustums of inverted pyramids (FIG. 5).

Plurality of micropatterned features 30 may be arranged in any way; however, more efficient arrangements may be desirable when seeking to maximize the density of micropatterned features on a surface (of a definite surface area). In one embodiment, plurality of micropatterned features 30 are arranged in rows and columns. In one embodiment, plurality of micropatterned features 30 are arranged in contiguous rows and columns (e.g. a grid when viewed from the top).

In one embodiment, spacing between adjacent individual features (e.g microwells) is minimized. A relatively large space (i.e. non-minimal) between adjacent individual features (e.g microwells) leads to inefficiencies when an interest is to maximize scale of cultures, experiments, or assays. In one embodiment, spacing between adjacent of the plurality of micropatterned features 30 is minimized. In the context of a cell culture device, when the spacing between adjacent individual features is the diameter of a cell or greater, some cells in chamber 5 may not be deposited in the feature, but may rather rest on the spacing. In one embodiment, ridges between adjacent individual features (e.g microwells) are less than the diameter of a cell (e.g. <15 μm, <10 μm, <5 μm, <3 μm, <2 μm, or <1 μm). In one embodiment, adjacent ones of the plurality of micropatterned features are separated by an equal pitch.

In the context of device 1 used for cell culture, it may be desirable to limit at least the length and width of device 1 to ANSI pate formats.

The dimensions of each one of the plurality of micropatterned features 30 is not particularly limited. In one embodiment, each one of the plurality of micropatterned features 30 is dimensioned to receive more than one cell. In some embodiments, each one of the plurality of micropatterned features 30 are dimensioned to receive up to 100 cells. In one embodiment, each one of the plurality of micropatterned features 30 are dimensioned to receive up to 1000 cells. In one embodiment, each one of the plurality of micropatterned features 30 are dimensioned to receive up to 5000 cells. In one embodiment, each one of the plurality of micropatterned features 30 are dimensioned to receive up to 10000 cells. In one embodiment, each one of the plurality of micropatterned features 30 are dimensioned to receive more than 10000 cells.

In one embodiment, a depth MPa of each micropatterned feature is between about 50 μm to 4 mm. In one embodiment, a depth of each micropatterned feature is between about 75 μm to 3 mm. In one embodiment, a depth of each micropatterned feature is between about 100 μm to 2 mm.

In one embodiment, a width MP_{w} or diameter of each micropatterned feature taken in the plane across its opening is between about 50 μm to 5 mm. In one embodiment, a width or diameter of each micropatterned feature taken in the plane across its opening is between about 75 μm to 3 mm. In one embodiment, a width or diameter of each micropatterned feature taken in the plane across its opening is between about 100 μm to 2 mm.

In one embodiment, such as when a micropatterned feature is an inverted pyramid/cone or an inverted frustum of a pyramid/cone, a width of each micropatterned feature taken in the plane across its opening may be about 200 μm, and a depth of such micropatterned feature may be between about 100-150 μm. In a specific embodiment, the depth of such a micropatterned feature may be about 140 μm.

In one embodiment, such as when a micropatterned feature is an inverted pyramid/cone or an inverted frustum of a pyramid/cone, a width of each micropatterned feature taken in the plane across its opening may be about 400 μm, and a depth of such micropatterned feature may be between about 250-300 μm. In a specific embodiment, the depth of such micropatterned feature may be about 280 μm.

In one embodiment, such as when a micropatterned feature is an inverted pyramid/cone or an inverted frustum of a pyramid/cone, a width of each micropatterned feature taken in the plane across its opening may be about 800 μm, and a depth of such micropatterned feature may be between about 350-400 μm. In a specific embodiment, a depth of such micropatterned feature may be about 390 μm.

A relationship of MP and MP_{w} may be any ratio of the respective dimensions provided herein.

Notwithstanding the foregoing, the aspect ratio (that is, the ratio between the depth of and width across the opening of a micropatterned feature) is not particularly limited. In some embodiments, it may be desirable to minimize disruption of cells or aggregates within a micropatterned feature, thus an aspect ratio above 1, above 2, above 3 or above 4 may be preferred. However, the higher the aspect ratio the more difficult it may be to retrieve the contents of each micropatterned feature. In some embodiments, it may be desirable to prioritize recovery of cells or aggregates from within a micropatterned feature, thus an aspect ratio of about 1 or lower may be preferred.

In one embodiment, an aspect ratio of each micropatterned feature of plurality of micropatterned features 30 is 1, or lower than 1. In one embodiment, an aspect ratio of each micropatterned feature of plurality of micropatterned features 30 is between 0.5 and 1.

In embodiments of device 1 comprising plurality of micropatterned features (e.g. microwells), the number of individual such features is not particularly limited. Indeed, the number of individual features is constrained by their dimensions and by the dimensions of device 1, particularly bottom wall thereof. In embodiments, where device 1 occupies a footprint equivalent or substantially equivalent to a typical microplate (ANSI 1-2004, 127.76×85.48 mm) then it may be possible to provide >100,000 individual features, depending on their dimensions. For example, where a width of each pyramidal (or frustopyramidal) feature (taken across its opening) is 200 μm approximately 125,000 individual features may be provided in a single device 1, and where a width of each pyramidal (or frustopyramidal) feature (taken across its opening) is 400 μm approximately 35,000 individual features may be provided in a single device 1.

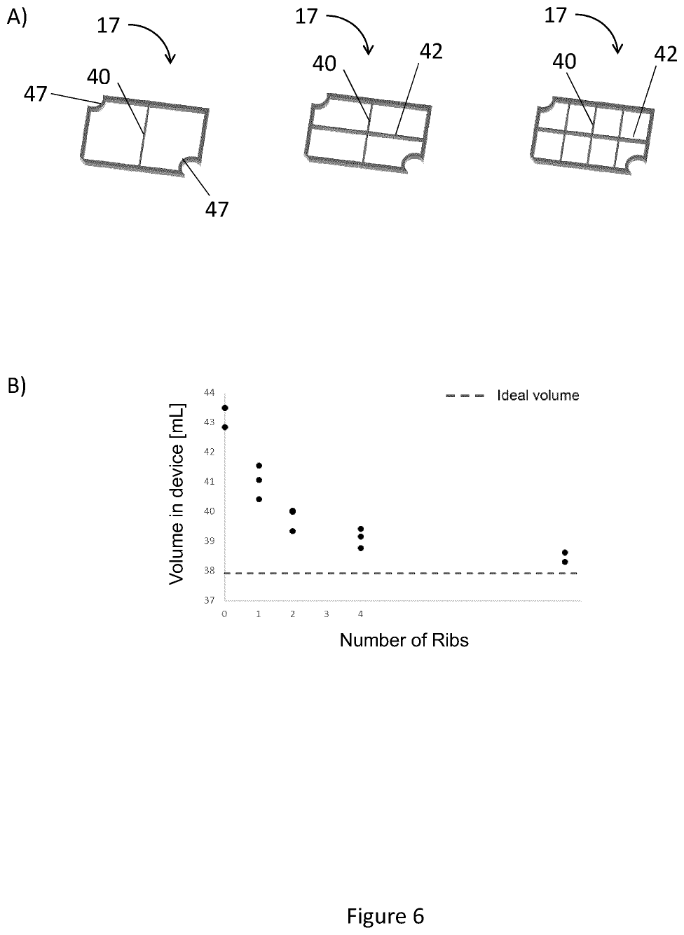

With reference to FIGS. 1, 3, 6 and 7, device 1 may further comprise frame 17 that is external chamber 5 that overlaps/overlies at least a perimeter of gas porous membrane 15. As indicated above frame 17 may facilitate attachment of gas permeable membrane 15 and housing 3. Frame 17 may also provide support to gas porous membrane 15, such as by limiting gas porous membrane stretch and corresponding chamber volume increase (when or as the chamber is filled with a fluid).

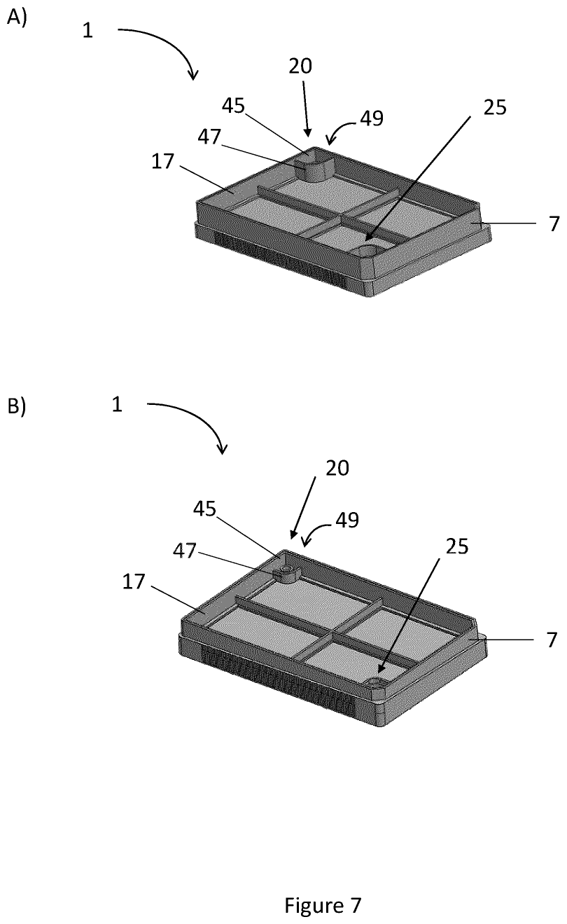

In one embodiment, frame 17 comprises at least one brace 40. In one embodiment, at least one brace 40 spans opposed or adjacent edges of frame 17 and overlies (or underlies depending on device 1 configuration) gas permeable membrane 15. In one embodiment, device 1 comprises a second brace 42. In one embodiment, second brace 42 spans the same or different opposed or adjacent edges of frame 17 and overlies (or underlies depending on device 1 configuration) gas permeable membrane 15. In one embodiment, second brace 42 intersects first brace 40. In one embodiment, second brace 42 does not intersect first brace 40. In embodiments of device 1 comprising at least one brace (and optionally second brace), the braces may support gas permeable membrane 7 and protect its integrity. For example, when gas permeable membrane 7 forms a top wall of chamber 5 it may bubble (expand upward) due to the force of liquid filling chamber 5 thereby creating localized liquid (e.g. cell culture medium) height difference across chamber 5 and potentially non-uniform waste, oxygen, nutrient and/or growth factor gradients. Indeed, FIG. 7 demonstrates that as the number of braces decreases, the volume of liquid in chamber 5 increases.

Frame 17 may be made of any material, but preferably comprises a polymer. In one embodiment, frame 17 is made of a material amenable to molding technology, such as injection molding. Non-limiting examples of materials that frame 17 may be made of include: PS, PMP, PC, PMMA, SBS/SEBS, silicon, silicone-based material, or a copolymer, such as a styrene block copolymer.

In one embodiment, membrane 15, frame 17, and housing 3 are made of the same material. In one embodiment, membrane 15 is made of a different material compared to frame 17 and housing 3. In one embodiment, each of membrane 15, frame 17, and housing 3 are made of different material.

In one embodiment, frame 17 and/or braces 40, 42 are attached to gas permeable membrane 15 using an adhesive (e.g. adhesive 16b). The adhesive may be any type of adhesive, provided that it is capable of adhering the materials of frame 17 (and/or braces) and gas permeable membrane 15. In one embodiment, the adhesive is a double-sided tape. In one embodiment, the adhesive is a glue. In one embodiment, the adhesive used to attach frame 17 and gas permeable membrane 15 is the same as the adhesive used to attach shoulder 12 and gas permeable membrane 15.

The dimensions or thickness of at least one first brace 40 (and second brace 42 when present) against gas permeable membrane 15 may impact oxygen diffusion thereacross, or the distribution of diffused oxygen throughout chamber 5. In general, a more even distribution of oxygen is observed when the thickness of a surface of at least one first brace 40 (and second brace 42 when present) against gas permeable membrane 15 is millimeter-scale (e.g. between 1-10 mm).

In certain embodiments of device 1, first port 20 and second port 25 may be configured in or integral with frame 17, and thus traverse frame 17. In one embodiment, first port 20 and second port 25 are positioned in (and traverse) diagonally opposed corners or edges of frame 17 (FIGS. 1 and 7). In one embodiment, first port 20 and second port 25 are positioned integrally in (and traverse) diagonally opposed corners or edges of frame 17 (FIGS. 1 and 7). In one embodiment, first port 20 and second port 25 are attached to bores molded into diagonally opposed corners or edges of frame 17 (FIGS. 1 and 7).

Frame 17 may comprise a perimetric frame wall 45. In one embodiment, frame wall 45 has the same or substantially the same width as shoulder 12. In one embodiment, frame wall 45 has a smaller width than shoulder 12. In an embodiment where frame wall 45 has a smaller width than shoulder 12, the width deficiency may be compensated by a flange that is connected to frame wall 45 and extends orthogonally to overlap at least part or all of the width of shoulder 12.

When positioned on shoulder 12, frame wall 45 extends from shoulder 12 toward or to an apex of one or more sidewalls 7. In one embodiment, a height of frame wall 45 extends to an apex of one or more sidewalls 7 when frame 17 is positioned on shoulder 12. In one embodiment, a height of frame wall 45 does not extend to an apex of one or more sidewalls 7 when frame 17 is positioned on shoulder 12.

In embodiments where first port 20 and second port 25 are configured in or on frame 17, they may be positioned adjacent to opposed edges or corners of frame 17 (FIG. 7). Thus, first port 20 and second port 25 may be at least partially bounded by a portion of frame wall 45, and in such cases first port 20 and second part 25 may respectively be fully surrounded by a connecting wall 47 that cooperates with the portion of frame wall 45. Thus, first port 20 and second port 25 may respectively be surrounded by a frame wall portion 45 and a connecting wall 47 to form a port reservoir 49. Port reservoir 49 may help confine and/or direct fluids being introduced into or removed from chamber 5.

In one embodiment, a height of connecting wall portion 47 is the same or substantially the same as a height of frame wall 45. That is to say, an apex of connecting wall portion 47 is the same or substantially the same as an apex of frame wall 45. In one embodiment, a height of connecting wall portion 47 is lower or shorter than a height of frame wall portion 45 (FIG. 7). A height of connecting wall portion 47 that is lower or shorter than a height of frame wall portion 45 may advantageously reduce siphoning of liquid from port reservoir 49, or chamber 5 via port reservoir 49, and out of device 1.

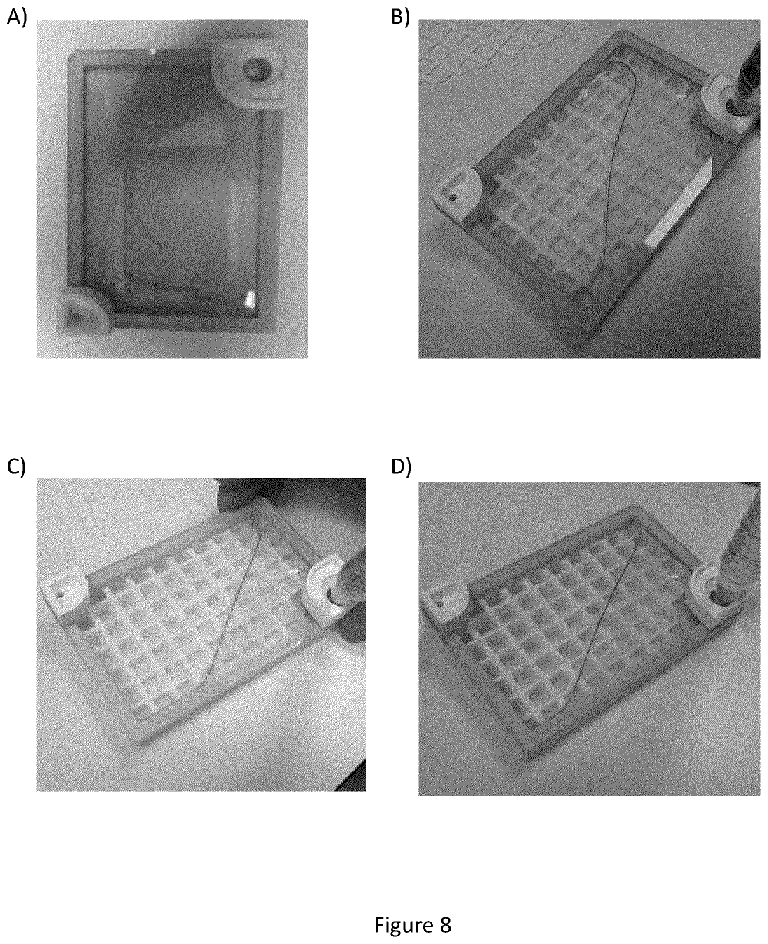

In use, a liquid (e.g. a culture medium comprising a suspension of cells) may be introduced into chamber 5 via second port 25 (e.g. a liquid port). To avoid or limit the formation of air bubbles, first port 20 (e.g. a venting port) allows air to vacate chamber 5 as it is displaced by the liquid (FIG. 8). Thus, a minimum diameter of first port 20 as described above may be selected so that liquid does not create a seal across the bore via surface tension forces.

The inventors have also unexpectedly discovered numerous advantages of the disclosed devices 1. For example, in a closed (e.g. sealed) configuration of device 1, chamber 5 may be completely filled with liquid (e.g. a culture medium comprising a suspension of cells) to eliminate meniscus and air bubbles which could create localized oxygen gradients. The elimination of meniscus reduces or obviates imaging artifacts, and may also reduce mass transfer effects. Also, by completely filling chamber 5, sloshing and the consequent disruption of the chamber contents (e.g. cells or aggregates) may be reduced or eliminated. Further, by completely filling chamber 5 with a liquid, such as culture medium, the effects of circulation by convection may be reduced or eliminated. The absence of air bubbles or air space above a cell culture may also serve to maximize nutrient and growth factor availability, while providing uniform or substantially uniform oxygenation to the entire culture surface area. Last, by completely filling chamber 5 with culture medium, the frequency of media changes may be reduced.

While complete filling of chamber 5 provides numerous advantages, a filling process is non-trivial. Filling (via second port 25) may be enhanced by slightly tilting device 1 during fluid introduction and/or removal (FIGS. 8 and 9). Indeed, air bubbles may form in chamber 5 if it is in a level position (i.e. normal to the force of gravity) during fluid introduction and/or removal. In contrast, tilts of about 1°, about 2°, about 3°, about 4°, about 5°, about 6°, or more, reduce or limit air bubble formation during fluid introduction and/or removal. In one embodiment, bottom wall of device 1 is tilted by more than 2°, more than 2.5°, or by more than 3° when it is supported by an angled surface (such as lid 60, as described below).

In one embodiment, bottom wall of chamber 5 may itself be manufactured with a slight grade or angle. In such an embodiment, the top wall of chamber 5 may also be correspondingly graded or angled.

In one embodiment, bottom wall of chamber 5 is inclined or elevated at a first corner or edge relative to an opposed second corner or edge during fluid introduction and/or removal. In one embodiment, the second corner or edge is diagonally opposed to the first corner. During fluid introduction, elevated first corner or edge may correspond to the location of second port 25, and during fluid removal elevated first corner or edge may correspond to the location of first port 20.

In one embodiment, device 1 may further comprise a lid 60 (FIGS. 9 and 10). Lid 60 may be manufactured to impart a tilt or angle when bottom wall of chamber/receptacle 5 rests on or is supported by an upper plane 62 of lid 60 (when lid 60 is situation on a level surface, e.g. a surface in the plane that is normal to the force of gravity). Thus, lid 60 can double as a platform for supporting device 1, such as during fluid introduction and/or removal. As described above, bottom wall of chamber/receptacle 5 is inclined when resting against or supported by a first corner or edge 63 of lid 60 and an opposed second corner or edge 64 of lid 60 during fluid introduction and/or removal.

Lid 60 may comprise a skirt 65 extending orthogonally downward from upper plane 62. In one embodiment, skirt 65 extends continuously around or about a perimeter of upper plane 62. In one embodiment, a height of skirt 65 is constant, that is to say that skirt 65 extends orthogonally downward the same distance from upper plane 62 at any point thereof. In one embodiment, a height of skirt 65 is non-constant, that is to say that skirt 65 does not extend the same distance orthogonally downward from upper plane 62.

In one such embodiment, a height of skirt 65 at first edge or corner 63 of lid 60 is minimal and a height of skirt 65 at opposed second edge or corner 64 of lid 60 is maximal. In one embodiment, a height of skirt 65 gradually changes, proceeding from first edge or corner 63 to opposed second edge or corner 64 of lid 60 (in both directions). Thus, when skirt 65 rests on a level surface (e.g. one in the plane normal to the force of gravity) upper plane 62 of lid 60 is tilted or at an angle relative to the surface.

Regardless of whether lid 60 is capping housing 3 or is supporting housing 3 first corner or edge 63 and second corner or edge 64 of lid 60 lie along a common axis ac with first port 20 and second port 25. However, in certain embodiments, when housing 3 is supported by lid 60 (e.g. when bottom wall of receptacle/chamber 5 is resting on upper plane 62 of lid 60), the bottom wall is tilted or inclined about a tilt axis aₜ that is orthogonal to the common axis. In one embodiment, tilt axis aₜ is orthogonal and intersects common axis aₜ. As described above, the bottom wall is tilted relative to a level surface, and the degree of tilt may be between 0 and 45 degrees. In one embodiment, the degree of tilt is less than 25°. In one embodiment, the degree of tilt is less than 15°. In one embodiment, the degree of tilt is less than 10°. In one embodiment, the degree of tilt is less than 5°. In one embodiment, the degree of tilt is between about 2° and 5°. In one embodiment, the degree of tilt is in the range of about 3°+1°.

In the context of lid 60 that is dimensioned to cover a standard ANSI plate: a tilt angle of 1° creates a roughly 2.5 mm height difference of skirt 65 between first corner or edge 63 and second corner or edge 64; a tilt angle of 2° creates a roughly 5 mm height difference of skirt 65 between first corner or edge 63 and second corner or edge 64; and a tilt angle of 3° creates a roughly 7.5 mm height difference of skirt 65 between first corner or edge 63 and second corner or edge 64.

When lid 60 is a cover or cap, an underside of upper plane 62 and/or skirt 65 may rest against one or more limits 70. In one embodiment, one or more limits 70 are situated around a periphery of one or more sidewalls 7 (on the opposite side of one or more sidewalls 7 relative to where shoulder 12 is situated). In embodiments where one or more limits 70 are situated around a periphery of one or more sidewalls 7, one or more limits 70 may be continuous about the perimeter or may be a plurality of distinct elements. In one embodiment, one or more limits are the apex (or rim) of one or more sidewalls 7 and an underside of upper plane 62 may rest thereupon. In one embodiment, lid 60 is supported by both the apex of one or more sidewalls 7 and by a continuous or a plurality of distinct features situated around a periphery of one or more sidewalls 7 (as described in foregoing).

In one embodiment, a height of one or more limits 70, such as second shoulder 72, is not constant, while a height of one or more sidewalls 7 may or may not be constant. In such embodiments, a height of one or more limits 70 is maximal at a first corner or edge 73 of housing 3 and a height of one or more limits 70 at an opposed second corner or edge 74 of housing 3 is minimal. In one embodiment, a height of one or more limits 70 gradually decreases (in both directions) from first corner or edge of housing 3 to the second corner or edge of housing 3.

In one embodiment, a contour of skirt 65 is complementary to a contour of one or more limits 70. In one embodiment a contour of the apex (or rim) of one or more sidewalls 7 is complementary to a contour of the underside of upper plane 62. In one embodiment, a contour of skirt 65 is complementary to a contour of one or more limits 70, and both the bottom wall of receptacle 5 and upper plane of lid 60 (when skirt 65 rests against one or more limits 70) lie in parallel planes which are normal to the force of gravity. In other words, bottom wall of receptacle 5 is level or substantially level when receptacle 5 rests on a level surface, and upper plane of lid 60 lies in a plane that is parallel to a plane of bottom wall when skirt 65 rests against one or more limits 70. Also in such embodiment, the bottom wall of receptacle 5 is tilted or inclined relative to the level surface when an underside of receptacle 5 (e.g. bottom wall) is positioned on the upper plane of lid 60 with skirt 65 against the level surface.

In one embodiment, device 1 comprises a standard lid 60, and further comprises a base adapter that provides a sufficient tilt of bottom wall of chamber/receptacle 5 for fluid introduction or withdrawal.

Device 1 may be sterile or sterilizable, such as by autoclaving, radiation, or alcohol treatment.

Device 1 may withstand centrifugation forces up to ˜10000×g, ˜5000×g, or ˜2500×g.

In another aspect, device 1 may comprise a) receptacle 5 having one or more sidewalls 7 extending substantially orthogonally upward from a bottom wall, b) one or more limits 70 (e.g. second shoulder) surrounding or about a periphery of one or more sidewalls 7 relative to receptacle 5, and c) skirted lid 60 wherein skirt 65 extends orthogonally downward from upper plane 62 of lid 60 (FIG. 10A and/or 10B).

In one embodiment, device 1 comprises a single receptacle. In one embodiment, device 1 comprises a plurality of receptacles (e.g. is a 6-, 12-, 24-, 48-, or larger well format microplate).

One or more limits 70 are essentially as described above. In some embodiments where one or more limits 70 is/are arranged around a periphery of one or more sidewalls 7, one or more limits 70 extend a distance (e.g. height) from bottom wall that is shorter relative to one or more sidewalls 7. In some embodiments, one or more limits 70 is a continuous feature around the periphery of one or more sidewalls 7, and may comprise a second shoulder 72 extending orthogonally or substantially orthogonally away from one or more sidewalls 7 relative to the inside of receptacle 5. In one embodiment, a height of one or more limits 70, such as second shoulder 72, is not constant, while a height of one or more sidewalls 7 may or may not be constant. In such embodiments, a height of one or more limits 70 is maximal at a first corner or edge 73 of receptacle 5 and a height of one or more limits 70 at an opposed second corner or edge 74 of receptacle 5 is minimal. In one embodiment, a height of one or more limits 70 gradually decreases (in both directions) from first corner or edge of receptacle 5 to the second corner or edge of receptacle 5.

Skirt 65 is essentially as described above. In one embodiment, skirt 65 is continuous, that is it bounds or extends around a perimeter of lid 60. A height of skirt 65 is not particularly limited. However, as described above, a height of skirt 65 at a first edge or corner 63 of lid 60 may be minimal and a height of skirt 65 at an opposed second edge or corner 64 of lid 60 may be maximal.

In one embodiment, a contour of skirt 65 is complementary to a contour of one or more limits 70, and both the bottom wall of receptacle 5 and upper plane of lid 60 (when skirt 65 rests against one or more limits 70) lie in parallel planes which are normal to the force of gravity. In other words, bottom wall of receptacle 5 is level or substantially level when receptacle 5 rests on a level surface, and upper plane of lid 60 lies in a plane that is parallel to a plane of bottom wall when skirt 65 rests against one or more limits 70. Also in such embodiment, the bottom wall of receptacle 5 is tilted or inclined relative to the level surface when an underside of receptacle 5 (e.g. bottom wall) is positioned on the upper plane of lid 60 with skirt 65 against the level surface.

As described above, an axis through the first edge or corner and the second edge or corner of lid 60 (e.g. the common axis) defines a slope (or direction of tilt/incline) of bottom wall of receptacle 5, and an axis that is orthogonal to the common axis (e.g. tilt axis) defines the axis about which bottom wall is tilted or inclined. Thus, the direction and amplitude (e.g. degree) of tilt/incline influence a travel of liquid along bottom wall of receptacle 5. The degree of tilt or incline about the tilt axis may be as described above.

In one embodiment, device 1 further comprises gas permeable membrane 15. As described above, and incorporated herein by reference, gas permeable membrane may be sealingly secured to one or more sidewalls 7.

Also as described above, and incorporated herein by reference, gas permeable membrane 15 may form the bottom wall of receptacle 5, or may be spaced apart from and lie in a plane parallel to a plane of the bottom wall.

Also as described above, and incorporated herein by reference, plurality of micropatterned features having any combination of the described characteristics may be formed in or on bottom wall of receptacle 5.

In one embodiment, device 1 further comprises first port 20 and opposed second port 25, each in fluid communication with chamber 5 formed between the bottom wall and gas permeable membrane 15, and circumscribed by one or more sidewalls 7.

Also as described above, and incorporated herein by reference, first port 20 and second port 25 may possess any combination of the described characteristics, including their relationship to frame 19.

### Methods

In another aspect of this disclosure are provided methods of using device 1, as disclosed above. Such methods may relate to assays using device 1. Such methods may further relate to culturing or incubating cells in a cell culture medium within device 1. Such methods may still further relate to aggregating and culturing cells within device 1. In some embodiments, devices 1 as disclosed above may be used in methods of culturing, incubating and/or aggregating cells under closed conditions; that is in the absence of direct contact with an environment external device 1.

Methods of culturing/incubating/aggregating cells in device 1 of this disclosure will require seeding cells suspended in a liquid, such as a culture medium, into receptacle/chamber 5. In one embodiment, cells are seeded via a port (e.g second port 25) in fluid communication with receptacle/chamber 5. In one embodiment, the receptacle/chamber 5 is completely filled with a liquid, such as a culture medium comprising a suspension of cells. In one embodiment, liquid is introduced by removing lid 60 and situating receptacle/chamber 5 at an angle (such as by resting housing 3 on lid 60, as described above), and discharging the liquid in the receptacle/chamber 5. The same steps may be performed for liquid withdrawal.

As described above, it may be necessary to tilt device 1 as a liquid is introduced into receptacle/chamber 5 via second port 25 (with first port 20 allowing for venting of displaced air). An angle of tilt or incline is not particularly limited, but is preferably between 0 and 45 degrees. In one embodiment, an angle of tilt or incline is below 25°. In one embodiment, an angle of tilt or incline is below 20°. In one embodiment, an angle of tilt or incline is below 15°. In one embodiment, an angle of tilt or incline is below 10°. In one embodiment, an angle of tilt or incline is between about 0 and 10°, between about 1 and 7°, between about 2 and 6°, or within the range between 3°±1°.

In one embodiment, as or after the liquid and particles (e.g. cells) suspended therein are introduced into receptacle/chamber 5, they are permitted to settle under the force of gravity against a bottom wall. In one embodiment, device 1 is removed from a tilted configuration and returned to a level configuration during the settling operation.

Once particles (e.g. cells) suspended in a liquid (e.g.culture medium) have been introduced into receptacle/chamber 5 they should be uniformly distributed in the liquid, and the particles/cells would be expected to settle at a substantially uniform density against bottom wall. If receptacle/chamber 5 is completely filled with the liquid, this should help minimize disruptive fluid forces within receptacle/chamber 5 that could result in a non-uniform distribution. Thus, in embodiments where bottom wall comprises a plurality of micropatterned features 30, each of such features would be expected to receive a uniform or substantially uniform number of particles/cells. In such case, following a sufficient incubation period the arising particle/cell aggregates would be expected to fall within a narrow or tight distribution of aggregate diameter.

In one embodiment, greater than 60% of arising aggregates have a diameter within +/−10% of the average aggregate diameter. In one embodiment, greater than 70% of arising aggregates have a diameter within +/−10% of the average aggregate diameter. In one embodiment, greater than 80% of arising aggregates have a diameter within +/−10% of the average aggregate diameter. In one embodiment, greater than 85% of arising aggregates have a diameter within +/−10% of the average aggregate diameter. In one embodiment, greater than 90% of arising aggregates have a diameter within +/−10% of the average aggregate diameter. In one embodiment, greater than 95% of arising aggregates have a diameter within +/−10% of the average aggregate diameter. In one embodiment, greater than 97% of arising aggregates have a diameter within +/−10% of the average aggregate diameter. In one embodiment, greater than 98% of arising aggregates have a diameter within +/−10% of the average aggregate diameter. In one embodiment, greater than 99% of arising aggregates have a diameter within +/−10% of the average aggregate diameter.

If the cells introduced and settled in receptacle/chamber 5 are adherent cells, but it is not desired that they adhere to bottom wall, then certain types of materials or treatments may be applied to at least bottom wall to prevent adhesion. In one embodiment, receptacle/chamber 5 may be prepared prior to seeding the cells, such as by coating with an anti-adherence rinse solution as commercialized by STEMCELL Technologies.

After the cells have been in culture/incubation/aggregation conditions for a sufficient period of time, the contents of receptacle/chamber 5 may be removed by tilting device 1 (at an angle as described above, such as in cooperation with lid 60) and removing fluid via second port 25.

In embodiments where device 1 comprises a plurality of micropatterned features 30, then liquid may be removed (as described above) with minimal or no disruption of the contents of the micropatterned features 30. In one embodiment, more than 80% of the liquid in receptacle/chamber 5 may be removed with minimal or no disruption to the contents of micropatterned features 30. In one embodiment, more than 85% of the liquid in receptacle/chamber 5 may be removed with minimal or no disruption to the contents of micropatterned features 30. In one embodiment, more than 90% of the liquid in receptacle/chamber 5 may be removed with minimal or no disruption to the contents of micropatterned features 30. In one embodiment, more than 95% of the liquid in receptacle/chamber 5 may be removed with minimal or no disruption to the contents of micropatterned features 30. In one embodiment, more than 97% of the liquid in receptacle/chamber 5 may be removed with minimal or no disruption to the contents of micropatterned features 30. In one embodiment, more than 98% of the liquid in receptacle/chamber 5 may be removed with minimal or no disruption to the contents of micropatterned features 30.

Once the liquid is removed from receptacle/chamber 5 fresh liquid may be added therein (as described above), or a particle/cell/aggregates harvest operation may be carried out. In one embodiment, the particle/cells/aggregates may be harvested from chamber 5 by adding a resuspension buffer or liquid, and agitating device 1 to resuspend the particles/cells/aggregates. In one embodiment, device 1 may be inverted and centrifuged to resuspend the particles/cells/aggregates. In one embodiment, a resupension buffer may be added to lift the particles/cells/aggregates off bottom wall, such as through buoyant forces.

In one embodiment, >50% of particles/cells/aggregates are recovered from receptacle/chamber 5. In one embodiment, >60% of particles/cells/aggregates are recovered from receptacle/chamber 5. In one embodiment, >70% of particles/cells/aggregates are recovered from receptacle/chamber 5. In one embodiment, >80% of particles/cells/aggregates are recovered from receptacle/chamber 5. In one embodiment, >90% of particles/cells/aggregates are recovered from receptacle/chamber 5. In one embodiment, >95% of particles/cells/aggregates are recovered from receptacle/chamber 5. In one embodiment, >97% of particles/cells/aggregates are recovered from receptacle/chamber 5. In one embodiment, >98% of particles/cells/aggregates are recovered from receptacle/chamber 5. In one embodiment, >99% of particles/cells/aggregates are recovered from receptacle/chamber 5.

Methods incorporating device 1 may yield clinical or sub-clinical quantities of cells/aggregates. In embodiments where device 1 is used to aggregate cells, the aggregates may exhibit a uniform or substantially uniform size distribution, as described above, lending themselves to downstream applications, including cell therapy application. If only a sub-clinical quantity of cells or aggregates are produced in a single device 1, the methods may involve a plurality of devices 1 to yield a clinical quantity of cell/aggregates.

In another aspect of this disclosure are provided methods of manufacturing device 1 of this disclosure. The methods will comprise the steps of forming housing 3, providing same, forming and/or providing gas permeable membrane 7, and assembling the various subcomponents to produce device 1 of this disclosure. Optionally, the methods may comprise forming, providing, and assembling frame 19 and/or lid 60.

The various subcomponents may be formed using any known process. In one embodiment, components are thermoformed, such as by liquid injection or liquid molding. In one embodiment, subcomponents are machined or milled.

In one embodiment, the various sub components are made of polymers or plastics.

In one embodiment, first port 20 and second port 25 are formed in a subcomponent, such as in frame 19. In one embodiment, first port 20 and second port 25 are formed separately from the subcomponents. In the latter embodiment, subcomponent that receives each of first port 20 and second port 25, such as frame 19 and/or housing 3 may need to be bored and optionally threaded in order to receive the port(s).

Sheets comprising plurality of micropatterned features (e.g. microwells) may be formed in various ways, including by: pouring PDMS over a mold and curing at an appropriate temperature (e.g. ˜90° C.) for an appropriate amount of time (e.g. ˜60 minutes); or by thermoformation, such as by hot-embossing, as depicted in FIG. 11.

In embodiments where plurality of micropatterned features 30 are formed in a bottom wall of chamber 5 that is not gas permeable membrane 15, they may be formed using any known process, including by liquid/injection molding, stamping, etching, hot-embossing, etc.

In embodiments where plurality of micropatterned features 30 are formed in a bottom wall of chamber 5 that is gas permeable membrane 15, they may be formed using any known process, including by thermoforming (e.g. molding, hot-embossing, etc).

In embodiments of hot-embossing methods of forming plurality of micropatterned features 30 in a polymer, the temperatures and pressures used to form plurality of micropatterned features 30 may depend on the polymer. For example, PS could withstand temperatures of approximately 90° C. and pressures of 1 MPa, PMP could withstand temperatures of approximately 150° C. and pressures of 5 MPa, PC could withstand temperatures of approximately 125° C. and pressures of 1 MPa, and SEBS could withstand temperatures of approximately 110° C. and pressures of 1 MPa. Nevertheless, optimization experiments were conducted that varied the time of pressing from 10 seconds to 2 minutes, and the pressure from 3 to 9 MPa. Still other optimization experiments were conducted that varied the pressing time from 5 to 30 seconds, the temperature from 120° C. to 170° C., and the pressure from 10 to 25 MPa.

In one embodiment, a pressing time of 5 seconds, a pressure of 17.5 MPa, and temperature of 170° C. or lower than 150° C. was used.

In one embodiment, embossing time plays an important role in low pressure embosses (<9MPa).

In one embodiment, a hot-embossing approach has a 15 minute cycle time. In one embodiment, where a cooling step was omitted, a cycle time could be reduced to approximately 15 seconds.

While FIG. 11 shows silicone used as a spacer, any appropriate spacer may be used, such as PMP or otherwise. In one embodiment, a cooling step may be omitted.

Once formed and/or provided, subcomponents of device 1 and gas permeable membrane 15 are assembled. In one embodiment, the subcomponents may be clamped to create leak-proof seal. In one embodiment, the components may be attached using fasteners such as screws or rivets. In one embodiment, the components may be welded, such as by ultrasonic welding. In one embodiment, the components may be bonded or adhered, such as by glues, tapes, or other adhesives. In one embodiment, a combination of any of the foregoing means of assembly may be used.

As mentioned above, ports may be separate subcomponents requiring further assembly or may be formed into a subcomponent of housing.

Regardless of the means used to assemble and secure subcomponents of device 1 to one another, it may be important that device 1 as a whole is biocompatible and non-toxic to cells or biomolecules that may be received through second port 25 and within chamber 5.

The above-described embodiments of the present disclosure are intended to be illustrative and in no way limiting. The embodiments are susceptible to many modifications. The invention and this disclosure are intended to encompass all such modifications within its scope, as defined by the claims, which should be given a broad interpretation consistent with the description as a whole.

1. A laboratory device, comprising
a housing having one or more sidewalls extending substantially orthogonally from a planar member;
a gas permeable membrane in a sealed engagement with the housing, the gas permeable membrane and the housing forming a receptacle having a chamber defined by a top wall and a bottom wall that are connected and circumscribed by the one or more sidewalls; and
a first port and an opposed second port each in fluid communication with the chamber,
wherein a diameter of the second port is the same or larger than a diameter of the first port.

2. The laboratory device of claim 1, further comprising a plurality of micropatterned features in the bottom wall of the chamber, wherein
a) the gas permeable membrane forms the bottom wall and the plurality of micropatterned features are formed in or on the gas permeable membrane, or
b) the gas permeable membrane forms the top wall and the planar member forms the bottom wall, and the plurality of micropatterned features are formed in or on the planar member.

3-4. (canceled)

5. The laboratory device of claim 1, wherein the first port and the second port extend through the top wall.

6. The laboratory device of any one of claim 1, wherein
a) a diameter of the first port is between about 3 mm and 5 mm, and/or
b) a diameter of the second port is between about 3 mm and 12 mm.

7-8. (canceled)

9. The laboratory device of claim 1, further comprising a frame external the chamber and overlapping at least a perimeter of the gas permeable membrane, optionally wherein the frame comprises at least one brace against the gas permeable membrane to limit gas permeable membrane stretch and chamber volume increase when the chamber is filled with a fluid.

10. (canceled)

11. The laboratory device of claim 9, wherein the first port and the second port traverse opposed corners or edges of the frame.

12-13. (canceled)

14. The laboratory device of claim 1, further comprising a lid having a continuous skirt extending orthogonally downward from an upper plane thereof.

15. The laboratory device of claim 14, wherein a height of the skirt at a first edge or corner of the lid is minimal and a height of the skirt at an opposed second edge or corner of the lid is maximal.

16. The laboratory device of claim 15, wherein the first edge or corner of the lid, the second edge or corner of the lid, the first port, and the second port lie along a common axis when viewed from above and when the lid is in a position covering the housing, and wherein the bottom wall of the receptacle is tilted when the housing is positioned on the lid and when the skirt rests on a level surface.

17. (canceled)

18. The laboratory device of claim 16, wherein the bottom wall is tilted about a tilt axis that is orthogonal to the common axis.

19-21. (canceled)

22. The laboratory device of claim 1, wherein
a) a depth of each micropatterned feature is between about 100 μm to 4 mm, and/or
b) a width or diameter of each micropatterned feature taken in the plane across an opening thereof is between about 100 μm to 5 mm, and/or
c) an aspect ratio of each micropatterned feature is 1.

23-24. (canceled)

25. A laboratory device, comprising:
a receptacle having one or more sidewalls extending substantially orthogonally upward from a bottom wall;
one or more limits surrounding the one or more sidewalls, the one or more limits extending a non-constant and shorter distance from the bottom wall relative to the one or more sidewalls; and
a lid having a continuous skirt extending orthogonally downward from an upper plane thereof, a height of the skirt at a first edge or corner of the lid is minimal and a height of the skirt at an opposed second edge or corner of the lid is maximal,
wherein the bottom wall lies in a substantially level plane when the receptacle rests on a level surface, and the upper plane of the lid lies in a plane that is parallel to the bottom wall when the skirt rests against the one or more limits, and
wherein the bottom wall is tilted relative to the level surface when an underside of the receptacle is positioned on the upper plane of the lid as the skirt rests against the level surface.

26. The laboratory device of claim 25, wherein the bottom wall is tilted about a tilt axis that is orthogonal to an axis through the first edge or corner and the opposed second edge or corner of the lid.

27. (canceled)

28. The laboratory device of claim 25, further comprising a gas permeable membrane sealingly secured to the one or more sidewalls, wherein
a) the gas permeable membrane forms the bottom wall and the plurality of micropatterned features are formed in or on the gas permeable membrane, or
b) the gas permeable membrane forms the top wall and the planar member forms the bottom wall, and the plurality of micropatterned features are formed in or on the planar member.

29. (canceled)

30. (canceled)

31. The laboratory device of claim 25, further comprising a plurality of micropatterned features in the bottom wall of the receptacle.

32. (canceled)

33. The laboratory device of claim 31, wherein
a) a depth of each micropatterned feature is between about 100 μm to 4 mm, and/or
b) a width or diameter of each micropatterned feature taken in the plane across an opening thereof is between about 100 μm to 5 mm, and/or
c) an aspect ratio of each micropatterned feature is 1.

34-35. (canceled)

36. The laboratory device of claim 28, further comprising a first port and an opposed second port each in fluid communication with a chamber formed between the bottom wall and gas permeable membrane, and circumscribed by the one or more sidewalls.

37. The laboratory device of claim 36, wherein a diameter of the second port is the same or larger than a diameter of the first port.

38. The laboratory device of claim 36, further comprising a frame external the chamber and overlapping at least a perimeter of the gas permeable membrane, optionally wherein the frame comprises at least one brace against the gas permeable membrane to limit gas permeable membrane stretch and chamber volume increase when the chamber is filled with a fluid.

39. (canceled)

40. The laboratory device of claim 38, wherein the first port and the second port traverse opposed corners or edges of the frame.

41-42. (canceled)
